(19) United States
(12) Patent Application Publication
(10) Pub. No.: US 2010/0331604 A1
(43) Pub. Date: Dec. 30, 2010

(54) TREATMENT APPARATUS

(75) Inventors: Kazuo OKAMOTO, Ashigarakami-gun (JP); Takeo Ishii, Naka-gun (JP); Masahiro Onoda, Ashigarakami-gun (JP); Yukinori Kubotera, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Shibuya-ku (JP)

(21) Appl. No.: 12/874,386

(22) Filed: Sep. 2, 2010

Related U.S. Application Data
(63) Continuation of application No. PCT/JP2009/053443, filed on Feb. 25, 2009.

(30) Foreign Application Priority Data
Mar. 26, 2008 (JP) 2008-081533

(51) Int. Cl. A61N 2/02

(52) U.S. Cl. 600/13

(57) ABSTRACT

A treatment apparatus which applies a magnetic field of a proper magnitude to a specific place of a treatment object includes a magnetic field generation part provided with first and second core members having end faces arranged oppositely and radiating lines of magnetic force therebetween and coils at the bases of the first and second core members. Additionally, a space adjustment part adjusts a space between the end faces of the first core member and that of the second core member, and a control part controls energization of the coil according to the space between the end face of the first core member and that of the second core member, thereby adjusting the intensity of the magnetic field to be generated by the magnetic field generation part.

This application is a continuation of International Application No. PCT/JP2009/053443 filed on Feb. 25, 2009, and claims priority to Japanese Application No. 2008-081533 filed on Mar. 26, 2008, the entire content of both of which is incorporated herein by reference.

### TECHNICAL FIELD

The present invention generally relates to a treatment apparatus. More specifically, the invention pertains to a treatment apparatus having useful application in the medical field which produces a magnetic field of a proper intensity for application to a specific place of a treatment object.

### BACKGROUND DISCUSSION

In recent years, research has been conducted in relation to the application of magnetism to the medical field. Japanese Patent Laid-open No. Hei 7-171220 discloses an example of a magnetic stimulation apparatus for applying magnetism to a living body. The disclosed magnetic stimulation apparatus is configured to include a C-shaped magnetic member split at a substantially central portion, with the split magnetic bodies joined so that they can be opened and closed. With such a configuration, lines of magnetic force are radiated from one pole face to the other pole face, while varying the distance between the pole faces according to the size of a treatment object, whereby a magnetic field can be applied to the treatment object.

In the above-mentioned magnetic stimulation apparatus, however, the intensity of the magnetic field generated by the pole faces is constantly fixed. Therefore, there is the problem that it is impossible to apply a magnetic field of a proper magnitude to a specific place of the treatment object disposed between the pole faces. Application of surplus magnetic field to normal tissues of a living body may cause an excessive excitation of nerve tissues and alteration of nerves.

### SUMMARY

According to one aspect, a treatment apparatus includes a magnetic field generation part comprised of first and second core members each possessing a respective end face and a coil provided at a base portion of each of the first and second core members. The end faces of the first and second core members face one another to radiate lines of magnetic force between the end faces of the first and second core members. A space adjustment part adjusts a space between the end faces of the first and second core member, and a control part controls energization of the coils according to the space between the end faces of the first and second core members, to thereby adjust intensity of a magnetic field generated by the magnetic field generation part to treat the treatment part. The treatment apparatus disclosed here permits application of a magnetic field of a proper magnitude to a specific place of a treatment object, and allows the intensity of the magnetic field to be adjusted according to the space between the end faces of the first and second core members.

The core members are preferably arranged so that the axis of the tip end portion of the first core member and the axis of the tip end portion of the second core member are located on the same line. A magnetic field of proper intensity can thus be applied more accurately to a specific place of a treatment object.

The magnetic field control part controls the energization of the coils so that the intensity of the magnetic field at a middle portion between the end face of the first core member and the end face of the second core member will be a predetermined intensity, regardless of the space between the end faces of the first and second core members. A magnetic field of a proper or desired intensity can be applied to the central portion of a treatment object, regardless of the size of the treatment object.

The magnetic field control part controls the energization of the coils on a preliminarily determined relational expression showing the relation between the space between the end faces of the first and second core member and the magnitude of a current for the energization of the coils. The apparatus thus determines the magnitude of current corresponding to the space between the end faces of the first and second core members.

The treatment apparatus also includes a space detection part which detects the spacing between the end faces of the first and second core members, wherein the magnetic field control part controls the energization of the coils according to the space detected by the space detection part. The space between the end face of the first core member and the end face of the second core member can thus be detected relatively accurately.

The space adjustment part includes: a movable part provided at a base portion of at least one of the first and second core members; and a driving part which moves the movable part, thereby relatively moving the end face of the first core member and the end face of the second core member toward or away from each other. The space between the end face of the first core member and the end face of the second core member can thus be adjusted.

The space detection part can include a displacement sensor which detects a displacement of the movable part. The space between the end face of the first core member and the end face of the second core member can thus be detected relatively accurately.

In addition, the end faces of the first and second core members are preferably curved and possess a concave shape. This allows a more uniform magnetic field to be generated between the end faces of the first and second core members, as compared with a configuration in which the end faces of the core members are flat.

The treatment apparatus also includes elastic members which cover the end faces of the first and second core members, The treatment object can thus be held between the end face of the first core member and the end face of the second core member, without applying excessive pressure to the treatment object and without imparting a thermal burn to the treatment object. The end faces of the first and second core members and the treatment object can also be maintained out of contact with one another.

The treatment apparatus additionally includes a pressure detection part at the end face of at least one of the first and second core members and detects a pressure exerted on the end face, and a position adjustment part which controls the driving part so that the pressure on the end face detected by the pressure detection part will be a predetermined pressure. The apparatus desirably allows contact between the end face of the core member and the treatment object to be detected, and the core member can be put in contact with the treatment object with a predetermined pressure.

A distance detection part can be provided at the end face of at least one of the first and second core members to detect the distance between the end face and the treatment object, while a distance adjustment part controls the driving part so that the distance detected by the distance detection part will be a predetermined distance. Thus, in addition to the end face of the core member and the treatment object being maintained out of contact, the apparatus allows the distance between the end face of the core member and the treatment object to be kept at a predetermined distance.

The space adjustment part preferably holds the first and second core members with a force greater than a magnetic force acting between the end faces of the first and second core members. The space between the end faces of the first and second core members is thus inhibited or prevented from being changed by the magnetic force acting between the end faces of the first and second core members. And the treatment object can be inhibited or prevented from being pressed by the core member with an excessive force.

The coil associated with each core member can be a plurality of coils connected in parallel to a power supply and so a voltage to be impressed on or applied to the coils can be reduced.

The treatment apparatus can also be outfitted with an overheat sensor forming a first temperature detection part which detects the temperature of at least one of the first and second core members, and a cut-off circuit part which cuts off the energization of the coils when the temperature detected by the first temperature detection part exceeds a predetermined temperature. This allows detection of an abnormal heat generation at the coil so that operation of the treatment apparatus can be stopped.

A second temperature detection part can also be provided in the form of a temperature sensor which detects the temperature of at least one of the first and second core members. Additionally, a cooling device can be provided which cools the core member on the basis of the temperature detected by the second temperature detection part. This helps prevent a rise in the temperature of the first and second core members.

The magnetic field generated by the magnetic field generation part is a varying magnetic field having a frequency of 10 to 300 Hz, and the intensity of the magnetic field at the middle portion between the end faces of the first and second core members is in the range of 30 to 1000 mT. An AC magnetic field can thus be applied to the treatment object, and the AC magnetic field can be applied to a nerve present in an affected part, thereby suppressing pain at the affected part.

The space between the end faces of the first and second core member is preferably adjusted within the range of 20 to 200 mm and so treatment objects of various sizes can be held between the end face of the first core member and the end face of the second core member.

According to another aspect, a treatment apparatus includes first and second core members each made of magnetic material, with each of the first and second core members possessing an end face, with the end face of the first core member facing the end face of the second core member, with the end face of the first core member being spaced apart from the end face of the second core member so a spacing exists between the end face of the first core member and the end face of the second core member and so that a treatment part is positionable between the end face of the first core member and the end face of the second core member. At least one first coil surrounds the first core member and at least one second coil surrounds the second core member to generate a magnetic field when current is supplied to the at least one first coil and the at least one second coil. The first and second core members are relatively movable to adjust the spacing between the end face of the first core member and the end face of the second core member. A motor is connected to at least one of the first and second core members to effect the relative movement of the first and second core members to adjust the spacing between the end face of the first core member and the end face of the second core member, and a power supply is connected to the at least one first coil and to the at least one second coil to supply current to the at least one first coil and to the at least one second coil. A controller is connected to the motor to control the motor and adjust the spacing between the end face of the first core member and the end face of the second core member. The controller is also connected to the power supply to control the current supplied by the power supply to the at least one first coil and to the at least one second coil to adjust the intensity of the magnetic field to be generated for treating the treatment object.

Another aspect involves a method of treating a treatment object. The method includes positioning the treatment object between an end face of a first core member and an end face of a second core member, wherein the end face of the first core member and the end face of the second core member face one another with a spacing between the end face of the first core member and the end face of the second core member, and with at least one coil surrounding the first core member and at least one coil surrounding the second core member. The method also involves determining the spacing between the end face of the first core member and the end face of the second core member, determining the amount of current to apply to the at least one coil surrounding the first core member and the at least one coil surrounding the second core member, using the determined spacing, so that a magnetic field of predetermined intensity will be applied to a central portion of the treatment object, and applying the amount of current to the at least one coil surrounding the first core member and the at least one coil surrounding the second core member while the treatment object is positioned between the end face of the first core member and the end face of the second core member to energize the at least one coil surrounding the first core member and the at least one coil surrounding the second core member and produce the magnetic field of predetermined intensity which is applied to the central portion of the treatment object.

### BRIEF DESCRIPTION OF THE DRAWING FIGURES

FIG. 1 is a perspective view of a treatment apparatus according to an embodiment disclosed here.

FIG. 2 is a block diagram schematically showing the configuration of the treatment apparatus shown in FIG. 1.

FIG. 3 is a flow chart illustrating a treatment (curing treatment) carried out by the treatment apparatus shown in FIG. 1.

FIG. 4 shows simulation results of a magnetic field generated by the treatment apparatus shown in FIG. 1.

FIG. 5(A) is a graph showing the relationship between the space between end faces of first and second core members and a current for energization of coils, and FIG. 5(B) is a graph showing the relationship between the space between the end faces of the first and second core members and electric power consumed in a magnetic field generation part.

FIGS. 6(A) and 6(B) show variations in the number of impulses of evoked action potential in the case where a nerve of a rat is electrically stimulated.

FIGS. 7(A) and 7(B) shows variations in the number of impulses of evoked action potential in the case where an AC magnetic field is applied to a nerve of a rat stimulated electrically.

FIG. 8 illustrates the influence of an AC magnetic field on a foot-lifting reaction threshold by carrageenan.

### DETAILED DESCRIPTION

Set forth below is a description of one embodiment of a treatment apparatus disclosed here. In the drawings, the same or equivalent features are denoted by the same reference symbols.

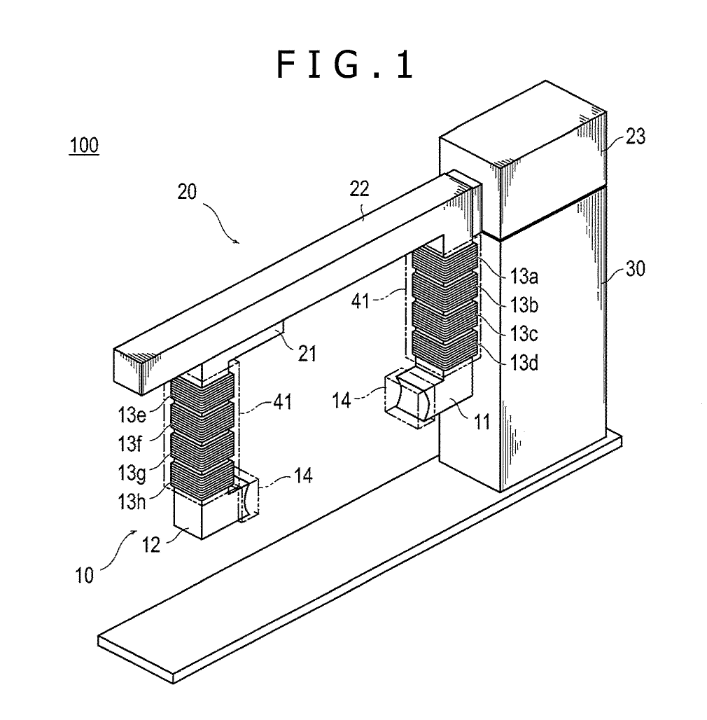

Referring to FIG. 1, the treatment apparatus in this embodiment is configured so that a treatment object part (e.g., arm) of a patient is held between a first core member and a second core member, whose pole faces for radiating lines of magnetic force therebetween are arranged oppositely, and a pulsed magnetic field or AC magnetic field of a predetermined intensity is applied to a central portion of the treatment object region.

As shown in FIG. 1, the treatment apparatus 100 in this embodiment includes a magnetic field generation part 10, a space adjustment part 20, and a control part 30.

### (Magnetic Field Generation Part)

The magnetic field generation part 10 generates a magnetic field to be applied to a treatment object part. The magnetic field generation part 10 has first and second core members 11 and 12 which are formed of a magnetic material, and coils 13 composed of electric wire wound around the base portions of the first and second core members 11 and 12.

The first and second core members 11 and 12 are each L-shaped, and have end faces arranged opposite one another (i.e., facing or opposing each other) so that the axes of their tip end portions which radiate lines of magnetic force coincide (are coincident with each other. That is the tip end portions of the first and second core members are coaxial with one another. The first and second core members 11 and 12 are formed from a magnetic material such as ferrites, soft iron, iron, silicon steel, permalloy, and amorphous-metal soft magnetic materials. First to fourth coils 13a, 13b, 13c and 13d are provided at a base portion of the first core member 11, while fifth to eighth coils 13e, 13f, 13g and 13h are provided at a base portion of the second core member 12. Channels 41 in which to form air streams for cooling the coils 13 are formed around the coils 13.

The end faces of the first and second core members 11 and 12 in this embodiment are each formed as arched concaved surfaces. The end faces formed as arched concaved surfaces are each covered with an elastic member (e.g., polyurethane) 14. In this embodiment, a pressure sensor is provided between the end face of the second core member 12 and the elastic member 14.

From the viewpoint of topically treating a specific place of a treatment object region, the width of the end face is preferably in the range of 10 to 50 mm. From the viewpoint of generating a more uniform magnetic field, the radius of curvature of the arched end face is preferably comparable to the width of the end face.

According to the magnetic field generation part 10 thus configured, energization of the coils 13 results in lines of magnetic force being radiated between the end face of the first core member 11 and the end face of the second core member 12.

### (Space Adjustment Part)

The space adjustment part 20 adjusts the space between the end face of the first core member 11 and the end face of the second core member 12. The space adjustment part 20 in this embodiment adjusts the space between the end faces of the first and second core members 11 and 12 by moving the second core member 12 toward or away from the first core member 11. The space adjustment part 20 includes a movable part 21 provided at a base portion of the second core member 12, a guide part 22 for guiding the movable part 21, and a driving part 23 for moving the movable part 21 along the guide part 22. The base portion of the first core member 11 is connected to the base portion of the guide part 22. The driving part 23 includes a ball screw and a motor. In addition, the driving part 23 is provided with a displacement sensor (e.g., a motor encoder) for detecting the displacement amount of the movable part 21.

According to the space adjustment part 20 thus configured, a movement of the movable part 21 along the guide part 22 causes the end face of the second core member 12 to be moved toward or away from the end face of the first core member 11. The space between the end face of the first core member 11 and the end face of the second core member 12 preferably has a movable range of 20 to 200 mm, from the viewpoint of holding the treatment object region between the end faces. In addition, the movable part 21 and the guide part 22 in this embodiment are each formed from a magnetic material, and they constitute a magnetic circuit together with the first and second core members 11, 12.

### (Control Part)

The control part or controller 30, as a magnetic field control part, controls the energization of the coils 13, thereby adjusting the intensity of the magnetic field to be generated by the magnetic field generation part 10. The control part 30 controls the magnitude of a current passed through the coils 13 (for example, the amplitude of a pulsed current) according to the space between the end face of the first core member 11 and the end face of the second core member 12 so that the intensity of the magnetic field at the middle point between the end face of the first core part 11 and the end face of the second core member 12 will be a predetermined intensity, independently from the space between the end faces of the first and second core members 11, 12.

The control part 30 in this embodiment, as a position adjustment part, adjusts the position of the second core member 12 by controlling the driving part 23 so that the end face of the second core member 12 is put into contact with the treatment object region through the elastic member 14 therebetween under a predetermined pressure.

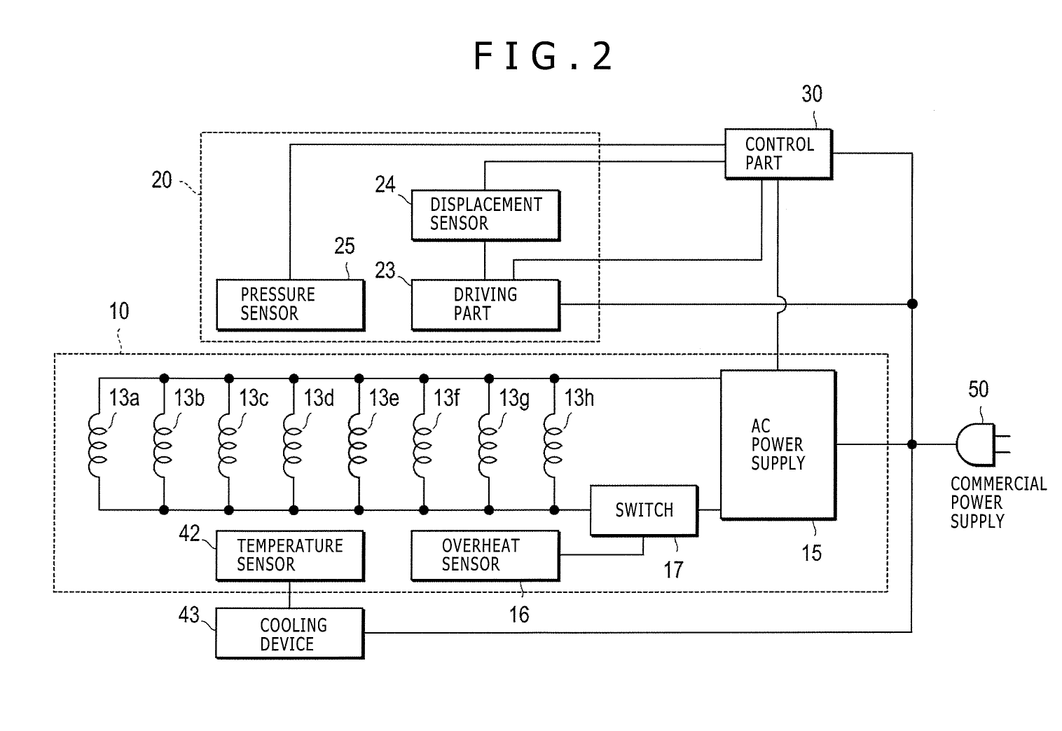

A schematic configuration of the treatment apparatus 100 in this embodiment is described in detail below with reference to FIG. 2 which is a block diagram showing schematically the configuration of the treatment apparatus shown in FIG. 1. As above-mentioned, the treatment apparatus 100 in this embodiment has the magnetic field generation part 10, the space adjustment part 20, and the control part 30.

As shown in FIG. 2, the eight coils 13a-13h provided at the base portions of the first and second core members 11 and 12 are connected in parallel to an AC power supply 15. The AC power supply 15 is supplied with electric power from a commercial power supply 50, and supplies a pulsed current or AC current to the coils 13. The AC power supply 15 is controlled by the control part 30 so as to supply the coils 13 with the pulsed current or AC current of a predetermined magnitude. The magnitude of the current is controlled by a control using an active element such as transistor, an inverter control system, or a control system using a transformer.

The control part 30 is electrically connected with a displacement sensor (e.g., a motor encoder) 24 provided in the driving part 23, which driving part 23 moves the second core member 12. The control part 30, receiving an output signal from the displacement sensor 24, computes the space between the end face of the first core member 11 and the end face of the second core member 12, and controls the magnitude of the current (or voltage) supplied to the coils 13 in such a manner that the intensity of the magnetic field at the middle point between the end face of the first core member 11 and the end face of the second core member 12 will be a predetermined intensity, independently of the space between the end faces.

In addition, the control part 30 is electrically connected with a pressure sensor 25 provided at the end face of the second core 12. The pressure sensor 25 is a distance detection part provided at the end face of the core to detect the distance between the end face and the treatment object. The control part 30 adjusts the positional relation between the end face of the second core member 12 and the treatment object region by controlling the driving part 23 so that the output of the pressure sensor 25 will be a fixed value.

The treatment apparatus 100 in this embodiment is provided with an overheat sensor 16 as a first temperature detection part for detecting the temperature(s) of the first and second core members 11, 12. In addition, between the coils 13 and the AC power supply 15, there is provided a switch 17 as a cut-off circuit part by which energization of the coils 13 is cut off when the temperature detected by the overheat sensor 16 reaches or exceeds an allowable value (e.g., 40° C.).

Further, the treatment apparatus 100 in this embodiment is provided with: a temperature sensor 42 as a second temperature detection part for detecting the temperature(s) of the first and second core members 11, 12; and a cooling device (e.g., cooling fan) 43 which receives a signal from the temperature sensor 42 and supplies air into the channels 41 provided around the coils 13, to thereby cool the first and second core members 11 and 12 and the coils 13. A water cooling system or a system of cooling the coils 13 by immersing them in an oil may be used as alternative cooling devices 43. As a further choice, intermittent generation of magnetism may be adopted so as to prevent an excessive rise in the temperature.

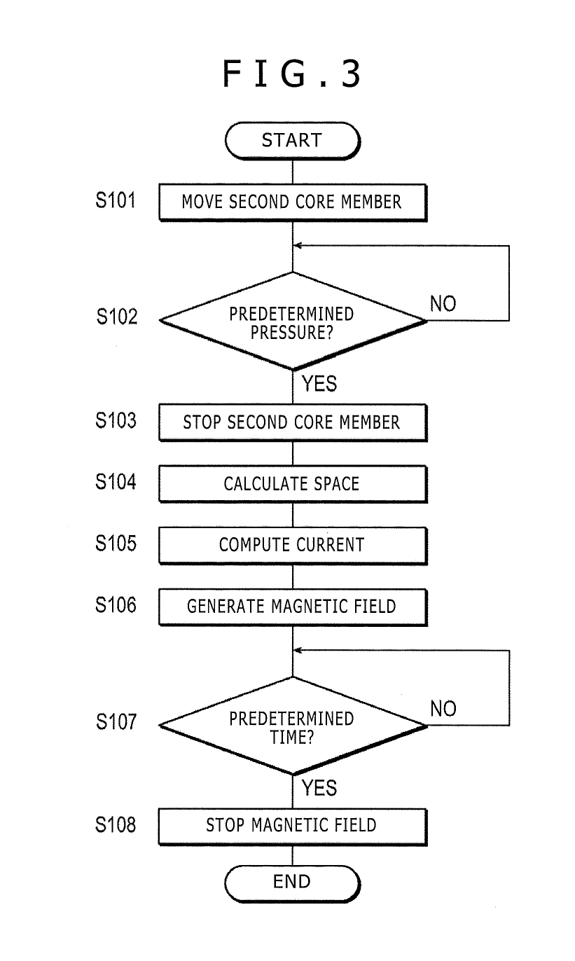

According to the treatment apparatus 100 in this embodiment configured as above, first a treatment object region is positioned or held between the first core member 11 and the second core member 12 through the elastic members 14. Next, a magnetic field is applied to the treatment object region while controlling the energization of the coils 13 so that the intensity of the magnetic field at the middle point between the end face of the first core member 11 and the end face of the core member 12 will be a predetermined intensity, independently of the spacing between the end faces of the first and second core members 11, 12. The treatment (curing treatment) carried out by the treatment apparatus 100 in this embodiment is described in detail below, referring to FIG. 3.

FIG. 3 is a flow chart illustrating a treatment which can be carried out by the treatment apparatus shown in FIG. 1. As has been described above, the treatment apparatus 100 in this embodiment controls the energization of the coils 13 according to the space between the end face of the first core member 11 and the end face of the second core member 12 so that a magnetic field of a predetermined intensity is applied to a central portion of a treatment object region. The magnetic field of the predetermined intensity in this embodiment means a magnetic field of an intensity which has been shown to be effective on the affected part.

As shown in FIG. 3, in the treatment in this embodiment, the treatment object region of a patient to be treated is first placed between the first core member 11 and the second core member 12, and the second core member 12 is moved toward the treatment object (step S101). In this embodiment, the driving part 23 moves the movable part 21 along the guide part 22, whereby the second core member 12 is moved toward the first core member 11.

Next, it is decided whether or not the pressure on the end face of the second core member 12 is a predetermined pressure (step S102). In this embodiment, the pressure on the end face of the second core member 12 is detected by the pressure sensor 25 provided at the end face of the second core member 12, for ensuring that the end face of the second core member 12 is put into contact with the treatment object region under the predetermined pressure. When the pressure on the end face of the second core member 12 is less than the predetermined pressure (step S102: NO), the second core member 12 is moved toward the treatment object region until the predetermined pressure is reached. On the other hand, when the pressure on the end face of the second core member 12 is not less than the predetermined pressure (step S102: YES), the movement of the second core member 12 is stopped (step S103).

Thus, according to the operations shown in steps S101 to S103, the treatment object region of the patient is held between the end face of the first core member 11 and the end face of the second core member 12. In this instance, clamping of the treatment object region under an excessive pressure is inhibited or prevented by the pressure sensor 25 provided at the end face of the second core member 12. Because the end faces of the first and second core members 11 and 12 in this embodiment are covered with the elastic members 14, the treatment object region is held between the end faces of the first and second core members 11 and 12 without receiving extreme pressure and/or heat from the end faces.

Subsequently, the space between the end face of the first core member 11 and the end face of the second core member 12 is computed (step S104). In this embodiment, the space between the end face of the first core member 11 and the end face of the second core member 12 is calculated, by the control part 30, from the output of the displacement sensor 24 provided in the driving part 23.

Next, a current corresponding to or associated with the thus calculated space is computed (step S105). In this embodiment, the magnitude of the current for the energization of the coils 13 is computed by the control 30 so that the intensity of the magnetic field at the middle point between the end faces of the first and second core members 11 and 12 will be a predetermined intensity, regardless of the spacing between the end faces. More specifically, the magnitude of the current corresponding to the space between the end faces that has been calculated in the treating operation shown in step S104 is computed by the control part 30, based on an end face space-current relational expression (translation table) showing the relation between the end face space and the current which has preliminarily been determined. Thus, regardless of the spacing between the end faces of the core members 11, 12, it is possible to produce a magnetic field intensity at the middle point between the end faces of the first and second core members 11, 12 that meets a predetermined intensity.

Then, the current of the thus computed magnitude is supplied to the coils 13, whereby a magnetic field is generated (step S106). In this embodiment, the AC power supply 15 is controlled by the control part 30 so that the current of the magnitude computed by the treating shown in step S105 flows in the coils 13, whereby lines of magnetic force are radiated from the end faces of the first and second core members 11, 12.

Thus, according to the operations shown in steps S104 to S106, the current of the magnitude corresponding to the space between the end face of the first core member 11 and the end face of the second core member 12 is supplied to the coils 13. As a result, a magnetic field of the predetermined intensity is generated at the middle point between the end face of the first core member 11 and the end face of the second core member 12, irrespective of the spacing between the end faces of the first and second core members 11, 12. In other words, a magnetic field of a predetermined intensity is applied to the central portion of the treatment object region, independently of the size of the treatment object region.

Subsequently, it is decided whether or not a predetermined time has passed (step S107). Then, at the time when the predetermined time has passed, the energization of the coils 13 is stopped, whereby the generation of the magnetic field is stopped (step S108), and the treatment (curing treatment) is finished.

As described above, in the treatment illustrated by the FIG. 3 flow chart, first a treatment object region of a patient is held between the end faces of the first and second core members 11, 12. Next, the space between the end face of the first core member 11 and the end face of the second core member 12 is calculated, and the magnitude of the current corresponding to the thus calculated space is computed. Then, the current of the thus computed magnitude is conveyed or passed to energize the coils 13, whereby a magnetic field of a predetermined intensity is applied to the central portion of the treatment object region, irrespective (independent) of the space between the end face of the first core member 11 and the end face of the second core member 12. In other words, regardless of the spacing or distance between the end face of the first core member 11 and the end face of the second core member 12, the magnetic field of predetermined intensity is applied to the central portion of the treatment object region.

In addition, in the treatment apparatus 100 according to this embodiment, a pulsed magnetic field or AC magnetic field is applied to the treatment object region, whereby nerves relating to pain are depressed, and the pain at the affected part is suppressed. Referring to the magnetic field to be generated by the magnetic field generation part, from the viewpoint of ensuring that a magnetic field of an intensity having been shown to be effective is applied to a specific place in the treatment object region, the intensity of the magnetic field at the middle point between the end faces of the first and second core members 11 and 12 (center magnetic flux density) is preferably in the range of 30 to 1000 mT, more preferably in the range of 50 to 300 mT. The frequency of the pulsed magnetic field or AC magnetic field, the intensity of which varies periodically, is preferably in the range of 10 to 300 Hz.

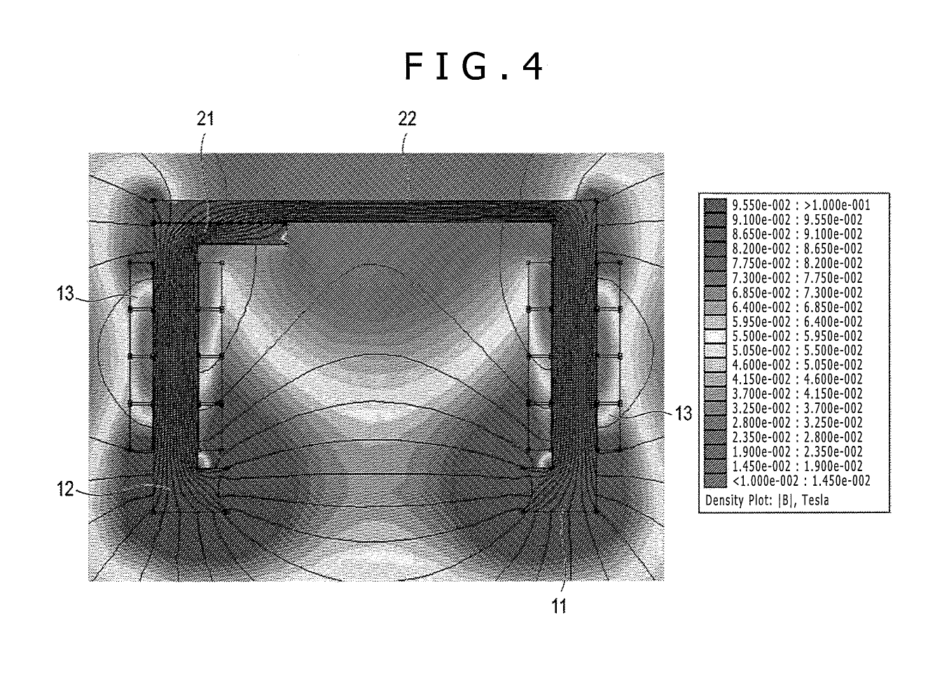

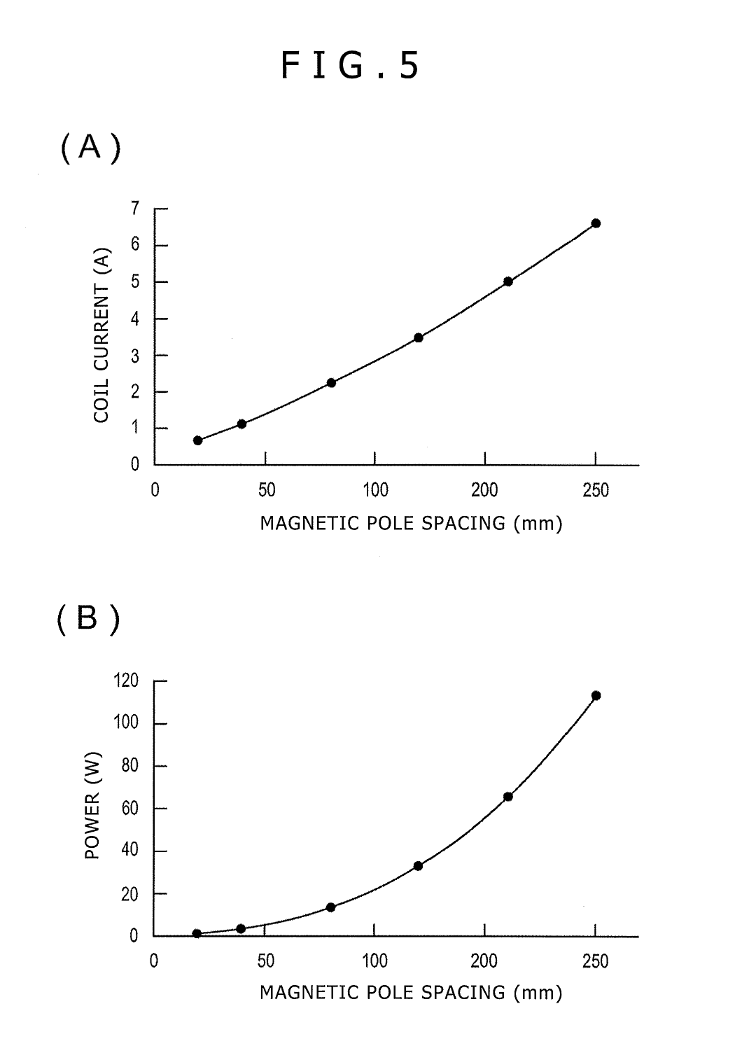

Now, referring to FIGS. 4 and 5, the magnetic field generated from the end faces of the first and second core members 11 and 12 of the treatment apparatus 100 in this embodiment is described below.

FIG. 4 shows simulation results of the magnetic field generated by the treatment apparatus shown in FIG. 1. In the simulation, the outside dimensions of the magnetic circuit constituted of the first and second core members 11 and 12, the movable part 21 and the guide part 22 were assumed to be 210 mm in height by 300 mm in length, the end face space was assumed to be 20 to 200 mm, and the profile of the first and second core members 11 and 12 in section perpendicular to the axis thereof was assumed to be a rectangular shape of 30 mm by 30 mm. In addition, the shape of that face of the movable part 21 which faces the guide part 22 was assumed to be a rectangular shape of 90 mm by 30 mm, and the space between the movable part 21 and the guide part 22 was assumed to be 0.2 mm, in computing the intensity distribution of the magnetic field.

As shown in FIG. 4, in the treatment apparatus 100 in this embodiment, lines of magnetic force are radiated between the end face of the first core member 11 and the end face of the second core member 12. The movable part 21 and the guide part 22 form the magnetic circuit, together with the first core member 11 and the second core member 12.

In the treatment apparatus 100 in this embodiment, energization of the coils 13 is controlled according to the spacing between the end face of the first core member 11 and the end face of the second core member 12 so that the intensity of the magnetic field (namely, the density of the lines of magnetic force) at the middle point between the end faces will be a fixed value, irrespectively of the space between the end faces. The amount of energization of the coils 13 such that the intensity of the magnetic field at the middle point between the end face of the first core member 11 and the second core member 12 will be 100 mT, irrespectively of the space between the end faces of the first and second core members 11 and 12, was computed while variously changing the space between the end faces of the first and second core members 11, 12. The computation results are shown in Table 1. Besides, graphic representations of the results given in Table 1 are shown in FIGS. 5(A) and 5(B).

TABLE 1

| Magnetic pole | Center magnetic | | | |
|---|---|---|---|---|
| spacing | field intensity | Power | Coil power | Coil current |
| (mm) | (mT) | (W) | (W) | (A) |
| 200 | 102 | 110.4 | 13.8 | 6.6 |
| 160 | 102 | 64 | 8 | 5 |
| 120 | 101 | 32 | 4 | 3.5 |
| 80 | 101 | 13.6 | 1.7 | 2.25 |
| 40 | 100 | 4 | 0.5 | 1.12 |
| 20 | 101 | 1.6 | 0.2 | 0.66 |

FIG. 5(A) shows the relationship between the spacing (distance) between the end faces of the first and second core members 11, 12 and the current for energizing the coils, whereas FIG. 5(B) shows the relationship between the spacing (distance) between the end faces of the first and second core members 11, 12 and the power consumed in the magnetic field generation part.

As shown in FIG. 5(A), the space between the end faces of the first and second core members 11 and 12 (magnetic pole spacing) and the magnitude of the current to be supplied to the coils 13 (coil current) for adjusting the intensity of the magnetic field to a fixed value exhibit a substantially proportional relationship. In other words, in order to cause the intensity of the magnetic field at the middle point between the end faces of the first and second core members 11 and 12 to be constant, the current supplied to the coils 13 must be greater as the space between the end faces of the first and second core members 11 and 12 increases. For example, to obtain a magnetic field intensity of 100 mT at the middle point between the end faces, a current of 0.66 A must be supplied to the coils 13 in the case of an end face spacing of 20 mm, and a current of 6.6 A must be supplied to the coils 13 in the case of an end face spacing of 200 mm.

In addition, as shown in FIG. 5(B), in the treatment apparatus 100 according to this embodiment, a greatest power of 110.4 W is consumed when the space between the end face of the first core member 11 and the end face of the second core member 12 (magnetic pole spacing) is 200 mm. This value does not mean a great power consumption, as compared with the power consumptions of ordinary household electric appliances. However, taking into account that substantially the whole of the electric power supplied to the coils 13 is converted into heat, it is expected that the power consumption at the coils 13 will be accompanied by considerable heat generation. Accordingly, it is preferable to provide the treatment apparatus 100 in this embodiment with the cooling device 43.

The simulation results show that in this embodiment of the treatment apparatus 100, a force of 13 N acts between the magnetic poles in the case where the space between the end face of the first core member 11 and the end face of the second core member 12 (magnetic pole spacing) is 200 mm. The space adjustment part 20 of the treatment apparatus 100 in this embodiment should thus have sufficient strength for holding the first and second core members 11 and 12 with a force greater than the attracting force of 13 N, for example.

As described above, with the treatment apparatus 100 in this embodiment, a pain at an affected part can be effectively mitigated by applying a magnetic field of a predetermined intensity to the affected part in a localized manner. In addition, because surplus application of a magnetic field to normal tissues can be minimized, excitation of normal nerves and alteration of nerves can be restrained. Particularly, in relation to various pains generated topically at movable regions such as articulations, e.g., articular rheumatism generated at a finger, an elbow, a knee, etc., osteoarthritis, low back pain arising from deformation of a vertebral body, etc. or fibromyalgia arising from a topical nerve alteration in the depth of a skin, painful diabetic neuropathy, complex regional pain syndrome (CRPS) and the like, the pain generated topically can be mitigated by efficient application of magnetism to the nerve relevant to the pain.

The treatment apparatus disclosed here has been described in the context of one disclosed and illustrated embodiment. Naturally, though, additions, modifications and omissions can be made by a person skilled in the art.

For instance, in the above-described embodiment, a configuration has been adopted in which the second core member is moved toward and away from the first core member which is in a fixed state. However, a configuration may be adopted in which both the first and second core members can be moved. In this case, it is preferable that the first and second core members are each provided with the pressure sensor. A configuration may also be adopted in which a non-contact type sensor is provided as a distance detection part, in place of the pressure sensor, and the control part controls the driving part so that the distance between the end face and the treatment object region will be a predetermined distance.

In addition, in the above-described embodiment, the core members possess a rectangular shape (profile) in cross-section perpendicular to the axis of the core members. However, the profile of the core members is not limited to the rectangular cross-sectional, but may be a circular or elliptical. Further, the shape of the end faces is not limited to the arched concave surface, but may be a spherical concave surface.

The treatment apparatus disclosed here will be discussed further with reference to an example, but it is understood that the invention is not limited to this example.

### (Experiment 1)

First, to verify the depressing effect of an AC magnetic field on nerves concerning a pain, the influence of an AC magnetic field on the activities of pain sensation nerves (C-fibers, Aδ-fibers) of the sciatic nerves of rats was tested.

As experimental animals, 6 to 8 weeks old crlj. WI rats (old name: crj:wistar) were purchased from Charles River Laboratories Japan. After an acclimation period of one week, the rats were subjected to experiments. The body weights of the rats at the time of the experiment were 270 to 370 g.

In an experimental procedure, the rats were first slightly sedated with ether in a draft, and then anesthetized by injecting about 1.1 to 1.3 g/kg of urethane into their abdominal cavities. More specifically, 1.1 mg/kg of a 20% solution of urethane was first injected into the abdominal cavities. Thereafter, a 40% (two-fold diluted concentration) solution of urethane was additionally injected in units of 0.05 mg/kg, according to the efficacy of anesthesia. This is because too large an amount of the anesthetic agent makes difficult the development of an evoked action potential from the sciatic nerves, whereas too small an amount of the anesthetic agent leads to a weaker anesthetic effect, rendering the respiration of the rats irregular and resulting in large scattering of the evoked action potential with time. After it was confirmed that the respiration of the rats had become stable and the anesthetic agent had taken an appropriate effect (respiration rate: 84 to 120/minute), the rats were held on a stationary base.

Next, the femoral skin of the rat was incised by surgical scissors to expose a muscle, and the muscle surface was shallowly cut open by the surgical scissors. Further, in order to minimize bleeding, while forcing open the slit in the muscle with forceps then on, without using the surgical scissors, the muscle was ripped. When the sciatic nerve was confirmed beneath the opening made by cutting, the cutting in the muscle was pinched with tweezers, and, in that condition, the sciatic nerve was carefully exfoliated from the surrounding connective tissues by use of minor forceps.

The action potential of the sciatic nerve of the rat was measured according to the method by Gokin et al of Harvard Medical School (Anestesiology, 95:1441-54, 2001). The method of Gokin et al is characterized in that a measurement object region is placed in a pool of liquid paraffin. Cotton yarns were tied to the four corners of the opening in the muscle, and while lightly pulling up the four cotton yarns, the cotton yarns were bound to a holding base fitted with two arms. As a result, a space was formed directly under the cutting in the muscle thus being pulled up, and a liquid paraffin (Kanto Chemical Co., Inc.) was poured so as to fill the space. The sciatic nerve was present in the manner of floating in the liquid paraffin.

Next, the sciatic nerve was hooked by bipolar electrodes (electrode spacing: 5 mm; Unique Medical Co., Ltd.) being hook-shaped at the tips, and the sciatic nerve was fixed to an electrode holding base capable of three-dimensional micro-movements in perpendicular directions, in the manner of lightly pulling up the nerve. In order to prevent the pool temperature from being lowered below 35° C. during the experiment, the temperature was constantly monitored by a thermocouple thermometer (CT-1307; CUSTOM), and the pool was warmed by a heat-radiating lamp (TECHNOLIGHT KTS-150RSV; Kenko), if necessary.

Recording of the action potential was conducted as follows. The hook-shaped bipolar electrode was used as a recording electrode, and an EEG electrode as a ground electrode was embedded beneath the skin of the region of chest. After the signal picked up was amplified by a factor of twenty thousands by a high-sensitivity bioelectric amplifier (ER-1 Extracelular Amplifier; CYGNUS TECHNOLOGY), the action potential waveform was displayed on the screen of a MACBook personal computer (Mac OSX version 10.4.9) through PowerLab 16/30 (AD INSTRUMENTS). To minimize the noises in the potential measurement, a low-pass filter and a high-pass filter of the high-sensitivity bioelectric amplifier were set to 3 kHz and 300 Hz, respectively.

Under urethane anesthesia, ordinarily, a spontaneous action potential from the sciatic nerve is not observed. In this experiment, in order to evoke an action potential in the sciatic nerve, a hindfoot of the rat was electrically stimulated. Stainless-steel disposable acupunctures (φ0.14 mm×40 mm; KANAKEN) were piercingly inserted into a skin between the second toe and the third toe and a skin between the fourth toe and the fifth toe, of one hindfoot (mainly left hindfoot) of the rat, as stimulating electrodes. The hindfoot of the rat was electrically stimulated from an electrical stimulation system (Model 238 High CURRENT SOURCE MEASURE UNIT; EKITHLEY) through Isolator (DSP-133B; DIA MEDICAL SYSTEM CO.). The electrical stimulus was a pulsed stimulus composed of five pulses with a frequency of 1 Hz, a pulse width of 1 ms, and an intensity of 5 to 15 mA. Such a pulsed stimulus was given repeatedly at intervals of 10 minutes.

Application of an AC magnetic field to the nerve will now be described below.

A magnetic field was applied to the rat by use of an AC magnetic field generation apparatus made by TERUMO Corporation on an experimental basis, or by use of a commercial 50-Hz magnetic field generation apparatus (AC magnetic field treatment apparatus; SOKEN MEDICAL Co., Ltd.). The former has a configuration in which an insulator-coated copper wire (diameter: 0.8 mm) is wound around a toroidal ferrite (outside diameter: 151 mm, inside diameter: 91.5 mm, thickness: 20 mm) having a 25 mm air gap. An AC current obtained by generating a sine wave by a function generator (WF1973; NF corporation) and amplifying the sine wave by PRECISION POWER AMPLIFIER 4502 (NF corporation) was supplied to the above-mentioned magnetic wave generation apparatus, whereby an AC magnetic field was applied to the rat.

The magnetic field application object region was a range from near the electrical stimulation object region to the heel, of the hindfoot of the rat, and AC magnetic fields of 50 Hz (1 to 17 mT), 1 kHz (1 to 10 mT) and 10 kHz (3 mT) were individually applied to the object region. The intensity of the magnetic field generated by the commercial magnetic field generation apparatus was about 50 mT. The magnetic field application time was set to be 20 minutes. Owing to the effect of the band pass filter at the frequency selected, no noise was generated during the action potential measurement in the case where the AC magnetic field was 50 Hz (1 to 17 mT). However, noises were generated in the cases where the AC current was 50 Hz (50 mT), 1 kHz, and 10 kHz; in these cases, therefore, the magnetic field application was stopped for several tens of seconds, during the action potential measurement. Incidentally, the magnetic field at the magnetic field application object region was measured by 5180 Gauss/Tesla Meter (TOYO Corporation).

A technique for evaluating the influence of the AC magnetic field on the nerve is described below.

In this experiment, the number of impulses in the evoked action potential was counted, to thereby evaluate the influence of the AC magnetic field on the nerve.

The number of impulses in the evoked action potential was counted after the experiment by an impulse measurement software Chart Pro Spike module (AD INSTRUMENTS). The number of impulses was counted for two groups (which are the pain sensation nerves), namely, the group of Aδ-fibers and the group of C-fibers. Whether the evoked potential was due to the M-fibers or due to the C-fibers was decided by the nerve conduction velocity. Gokin et al have reported that the nerve conduction velocities relevant to the Aδ-fibers and the C-fibers contained in the sciatic nerves of the rats are 2 to 10 m/s and 0.5 to 2 m/s, respectively. In this experiment, it was discriminated in which of the ranges reported by Gokin et al did the value obtained by dividing the distance between the stimulating electrode and the recording electrode by the time from the stimulation to the recording of the evoked potential (nerve conduction velocity) fall. Specifically, for example, in the case where the distance between the stimulating and recording electrodes was 10 cm, the evoked potential was attributed to the Aδ-fibers if the time elapsed from the stimulation to the recording of the evoked potential was in the range of 10 to 50 ms. On the other hand, the evoked potential was decided to be due to the C-fibers if the elapsed time was in the range of 50 to 200 ms. The results thus obtained were represented in terms of (mean)±(standard error), of the total number of impulses obtained through the five-pulse stimulation. For the evaluation of statistical significance, the Student's t-test (test of the mean obtained by a pair of samples) was used.

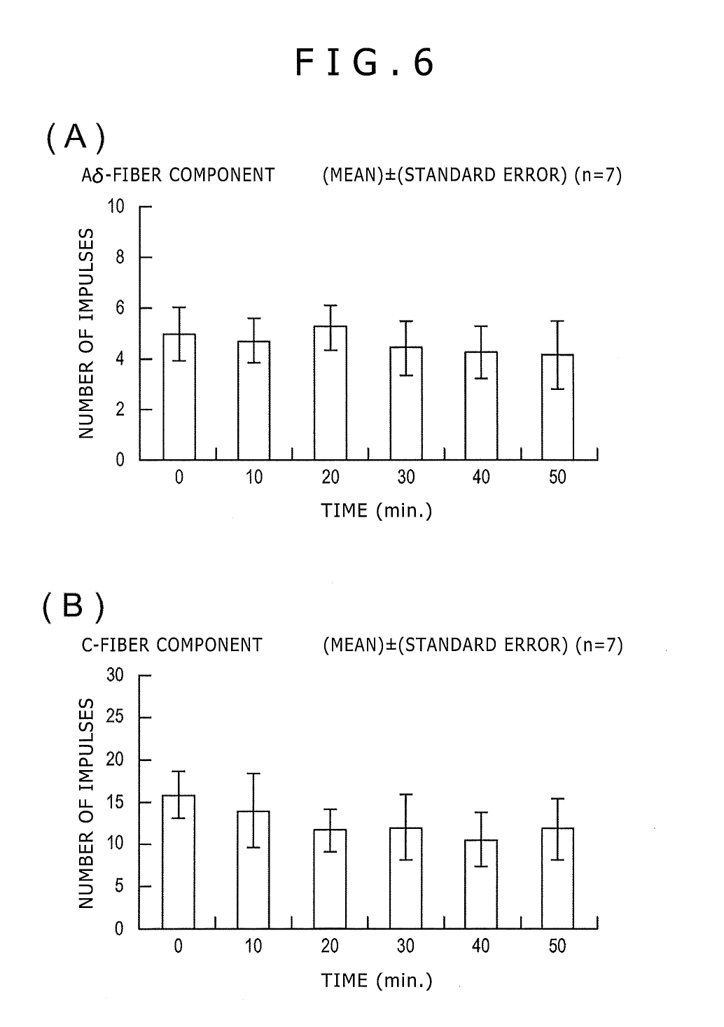

Upon electrical stimulation of the tiptoes of the rats (1 Hz, 1 ms, 5 to 10 mA, five pulses), the evoked action potential from the sciatic nerve was observed for substantially all the samples. The action potential was little recorded upon stimulation with one pulse, but the number of impulses was gradually increased starting from the stimulation with two pulses, and was maximized upon stimulation with 3 to 5 pulses; thus, a wind-up phenomenon was observed. Upon analysis of the action potential corresponding to the maximized number of impulses, 1 to 2 pulses of action potential seemingly due to firing from the A6-fibers were recorded, and, subsequently, a few pulses of action potential seemingly due to firing from the C-fibers were observed. When the electrical stimulation was repeated at intervals of 10 minutes, as shown in FIG. 6, both the A6-fiber component and the C-fiber component showed a comparatively stable reaction in which the number of impulses was gradually decreased.

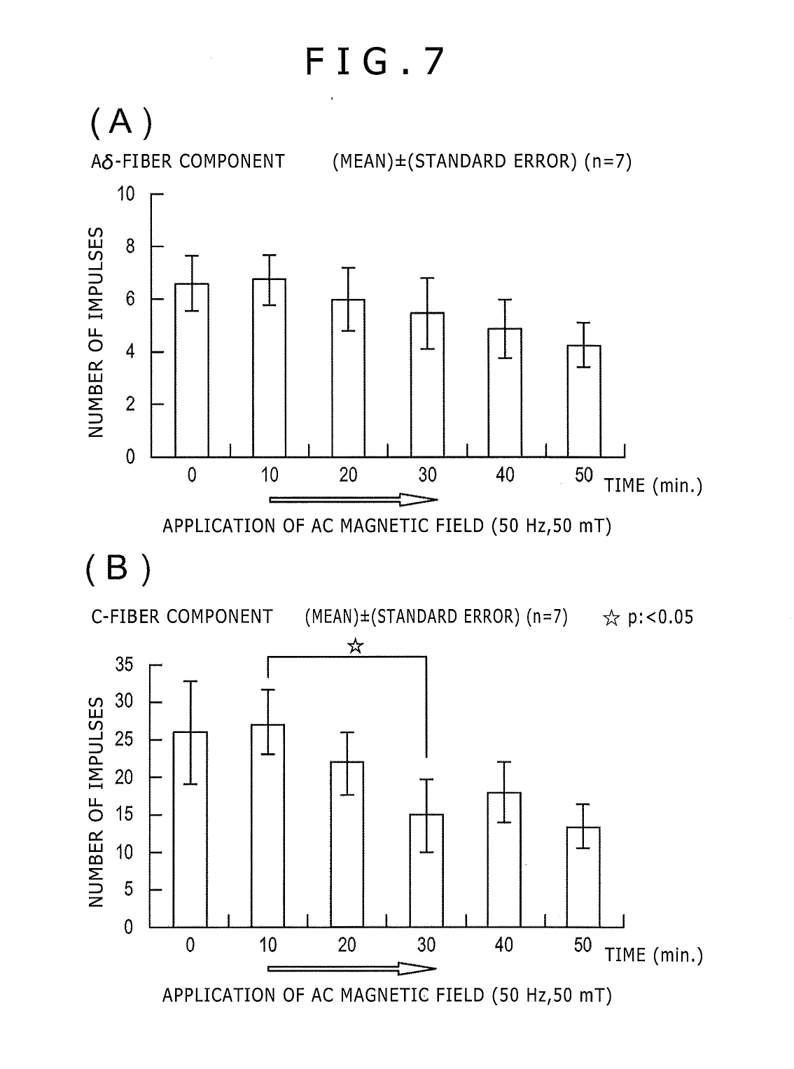

After the tiptoe was electrically stimulated twice at an interval of 10 minutes, an AC magnetic field of 50 Hz (5 mT, 17 mT, 50 mT), 1 kHz (10 mT), or kHz (3 mT) was applied for 20 minutes. As shown in FIG. 7(A), upon application of the magnetic field, little influence was observed on the action potential relevant to the Aδ-fiber component, in all the application conditions.

On the other hand, as shown in FIG. 7(B), regarding the C-fiber component, the number of impulses in the action potential was suppressed, even statistically significantly, by the application of the magnetic field of 50 Hz and 50 mT for 20 minutes. This suppression continued for a while, even after the application of the magnetic field was finished. In addition, the tendency toward a depressed action potential was observed also upon application of the magnetic field of 50 Hz and 30 mT. However, no clear effect was observed upon application of the magnetic field of 50 Hz and 5 mT. Further, no clear effect was observed either upon application of the magnetic field of 1 kHz (10 mT) or application of the magnetic field of 10 kHz (3 mT).

Thus, the depressing effects of AC magnetic fields on the nerve relating to pain were tested, and, as a result, it was confirmed that a depressing effect on the action potential of the pain sensation nerve (C-fibers) in the sciatic nerves of rats was attained when a magnetic field of 50 Hz and 50 mT was applied and when a magnetic field of 50 Hz and 30 mT was applied.

### (Experiment 2)

to verify the depressing effects of AC magnetic fields on the nerve relating to pain, influences of AC magnetic fields on edema at soles of rats were tested.

Specifically, by using foot edema rats with inflammation by carrageenan caused on their feet without anesthesia, influences of AC magnetic fields (50 Hz and 5 mT; 50 Hz and 50 mT; 1 kHz and 10 mT) on hyperalgesia were tested.

Crlj. WI rats (old name: crj:wistar) of 6 to 8 weeks old were purchased from Charles River Laboratories Japan, and, after an acclimation period of one week, they were served to an experiment. The body weights of the rats at the time of the experiment were 200 to 350 g.

Next, foot edema models of rats were produced. After sedating the rats with ether in a draft, 0.15 ml of an aqueous 1% solution of X-carrageenan was injected into the sole of one-side foot of each of the rats by hypodermic injection, to produce sole edema. Into the sole of the other foot of each of the rats, 0.15 ml of physiological saline was introduced by hypodermic injection.

The foot-lifting motion reaction threshold responding to a pressure stimulus was measured by placing the rat on a net stretched on a tetragonal wood frame (20 cm by 30 cm). As the net, one formed from racket gut (nylon mono-filament: 0.78 mm; GOSEN Co., Ltd.) and meshed in an about 1 cm width was used. The non-metallic net was used, based on a decision that the absence of metal between the apparatus and the rat would be preferable from the viewpoint of application of a magnetic field. The whole assembly was covered with a transparent plastic cage (12 cm by 20 cm by 11 cm in height) so that the rat cannot escape from on the net. Thereafter, the sole of the hindfoot of the rat was stimulated successively three times by perpendicularly pressing a von Frey filament (Touch Test; North Coast) against the sole from below the net. The stimulation time per run was set to be 3 seconds after the filament was pressed against the sole and curving of the filament was confirmed. In this case, the minimum pressure stimulus intensity of the von Frey filament that caused the rats to perform a foot-lifting action (foot-withdrawing reaction) two or more times was adopted as the reaction threshold (withdrawal pressure). The intensities of the von Frey filament used (or the forces with which the sole of the hindfoot of the rat was pressed) were eight kinds, namely, 60 g (5.88), 26 g (5.46), 15 g (5.18), 10 g (5.07), 8 g (4.93), 6 g (4.74), 4 g (4.56), and 2 g (4.31) (the numerical values inside the parentheses are logarithmic values).

For application of magnetism, AC magnetic fields of 50 Hz and 1 kHz were used. The intensity of the AC magnetic field was 5 mT or 50 mT for the 50 Hz, and 10 mT for the 1 kHz. The magnetic field application object region was set to be the carrageenan injection region of the hindleg, and the AC magnetic field was applied to the region from below the rat by a magnetism applying apparatus. In addition, the person in charge of the experiment changed the position of the netted frame so that the carrageenan injection region of the rat was always kept directly above the site where the magnetic flux density supplied from the magnetism applying apparatus was the strongest. The magnetic field application time was set to be 20 minutes immediately before injection of carrageenan, and, after the injection, 20 minutes immediately before each of the measurement times. Thus, where the measurement was continued for 6 hours after the carrageenan injection, the magnetic field application time was 140 minutes in total; where the measurement was continued for 3 hours after the injection, the magnetic field application time was 80 minutes in total.

The experiment was conducted for two rats a day. One of the two rats was subjected to application of the magnetic field, while the other was a control (magnetic field non-application) (Time matched control). The foot-lifting reaction in response to the pressure stimulus was measured, in principle, once before the carrageenan injection, and respectively 1 hour, 2 hours, 3 hours, 4 hours, 5 hours, and 6 hours after the injection (for some samples, the measurement was continued up to 3 hours after the carrageenan injection). The measurement results of the foot-lifting reaction thus obtained were presented in terms of (mean) (standard error), of the logarithm of the filament intensity (g). The volume of the foot edema was represented in units of ml, and presented in terms of (mean)±(standard error). For each of various times, whether or not there was a statistically significant difference between the control group and the magnetic field application group was evaluated by the Student's t-test (test by a mean of a pair of samples).

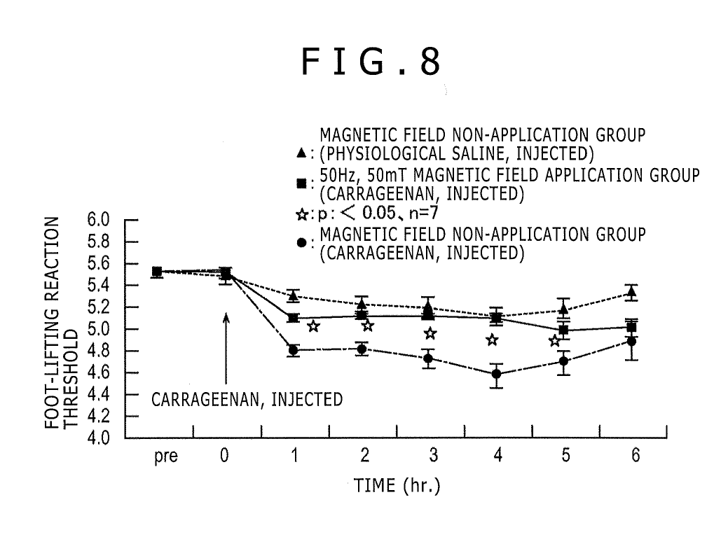

As shown in FIG. 8, for the group wherein an AC magnetic field of 50 Hz with an intensity of 50 mT was applied to the soles of the rats, the lowering in the foot-lifting reaction threshold due to the pressure stimulus at the carrageenan injection region was statistically significantly suppressed after 1 hour, 2 hours, 3 hours, 4 hours, and 5 hours, as compared with the magnetic field non-application group (p<0.05, n=7). On the other hand, at the magnetic field of 50 Hz and 5 mT and the magnetic field of 1 kHz and 10 mT, no suppression was observed (n=5).

Thus, by use of the rat foot edema, the depressing effect of AC magnetic fields on the nerve relating to pain were tested. As a result, it was confirmed that the nerve relating to pain is depressed when an AC magnetic field of 50 Hz is applied thereto in an intensity of 50 mT.

The detailed description above describes an embodiment of the treatment apparatus. However it is to be understood that the invention is not limited to the precise embodiment disclosed and variations mentioned. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A treatment apparatus comprising:
first and second core members each made of magnetic material, each of the first and second core members possessing an end face, the end face of the first core member facing the end face of the second core member, the end face of the first core member being spaced apart from the end face of the second core member so a spacing exists between the end face of the first core member and the end face of the second core member and so that a treatment part is positionable between the end face of the first core member and the end face of the second core member;
at least one first coil surrounding the first core member and at least one second coil surrounding the second core member to generate a magnetic field when current is supplied to the at least one first coil and the at least one second coil;
the first and second core members being relatively movable to adjust the spacing between the end face of the first core member and the end face of the second core member;
a motor connected to at least one of the first and second core members to effect the relative movement of the first and second core members to adjust the spacing between the end face of the first core member and the end face of the second core member;
a power supply connected to the at least one first coil and to the at least one second coil to supply current to the at least one first coil and to the at least one second coil;
a controller connected to the motor to control the motor and adjust the spacing between the end face of the first core member and the end face of the second core member; and
the controller connected to the power supply to control the current supplied by the power supply to the at least one first coil and to the at least one second coil to adjust an intensity of the magnetic field to be generated for treating the treatment object.

2. The treatment apparatus according to claim 1, further comprising a displacement sensor connected to the controller to provide input to the controller for determining the spacing between the end faces of the first and second core members, the controller controlling the power supply based on the determined spacing to supply the current to the at least one first coil and the at least one second coil.

3. The treatment apparatus according to claim 1, wherein the end face of each of the first and second core members is curved and possesses a concave shape.

4. The treatment apparatus according to claim 1, further comprising an elastic member covering the end face of the first core member and an elastic member covering the end face of the second core member.

5. The treatment apparatus according to claim 4, further comprising: a pressure sensor at the end face of at least one of the first and second core members to detect a pressure exerted on the end face of said at least one of the first and second core members, the pressure sensor being connected to the controller so the controller controls the motor to adjust the spacing between the end face of the first core member and the end face of the second core member so the pressure detected by the pressure sensor is a predetermined pressure.

6. The treatment apparatus according to claim 1, wherein the at least one first coil surrounding the first core member includes a plurality of first coils surrounding the first core member and connected in parallel to the power supply, and the at least one second coil surrounding the second core member includes a plurality of second coils surrounding the second core member and connected in parallel to the power supply.

7. A treatment apparatus which produces a magnetic field for treating a treatment object, the treatment apparatus comprising:
a magnetic field generation part comprised of first and second core members each possessing a respective end face and a coil provided at a base portion of each of the first and second core members, the end faces of the first and second core members facing one another to radiate lines of magnetic force between the end faces of the first and second core members;
a space adjustment part which adjusts a space between the end faces of the first and second core member; and
a control part which controls energization of the coils according to the space between the end faces of the first and second core members, to thereby adjust intensity of a magnetic field generated by the magnetic field generation part to treat the treatment part.

8. The treatment apparatus according to claim 7, wherein the first and second core members each possess a tip end portion terminating in the respective end face, the tip end portion of the first core member and the second core member are coaxial.

9. The treatment apparatus according to claim 7, further comprising a displacement sensor connected to the magnetic field control part to provide input to the magnetic field control part for determining the spacing between the end faces of the first and second core members, the control part controlling the energization of the coils based on the determined spacing to supply the current to the at least one first coil and the at least one second coil.

10. The treatment apparatus according to claim 7, wherein the space adjustment part includes a movable part provided at a base portion of at least one of the first and second core members, and a driving part connected to the movable part to move the movable part and thereby relatively move the end face of the first core member and the end face of the second core member toward or away from each other.

11. The treatment apparatus according to claim 10, further comprising a displacement sensor which detects a displacement of the movable part.

12. The treatment apparatus according to claim 10, further comprising a pressure sensor at the end face of at least one of the first and second core members to determine a distance between the treatment object and the end face of at least one of the first and second core members, the control part being connected to the pressure sensor to receive input from the pressure sensor, the control part being connected to the driving part to control the driving part and move the movable part based on the input received from the pressure sensor to apply a predetermined pressure to the treatment object.

13. The treatment apparatus according to claim 7, wherein the end faces of the first and second core members are curved and possess a concave shape.

14. The treatment apparatus according to claim 7, further comprising elastic members covering the end faces of the first and second core members.

15. The treatment apparatus according to claim 7, wherein plural coils connected in parallel to a power supply are provided at the base portion of each of the first and second core members.

16. The treatment apparatus according to claim 7, further comprising:
an overheat sensor which detects the temperature of at least one of the first and second core members; and
a switch which cuts off the energization of the coils when the temperature detected by the overheat sensor exceeds a predetermined temperature.

17. The treatment apparatus according to claim 7, further comprising:
a temperature sensor which detects temperature of at least one of the first and second core members; and
a cooling device which cools the at least one of the first and second core members based on the temperature detected by the temperature sensor.

18. A method of treating a treatment object comprising:
positioning the treatment object between an end face of a first core member and an end face of a second core member, the end face of the first core member and the end face of the second core member facing one another with a spacing between the end face of the first core member and the end face of the second core member, at least one coil surrounding the first core member and at least one coil surrounding the second core member;
determining the spacing between the end face of the first core member and the end face of the second core member;
determining an amount of current to apply to the at least one coil surrounding the first core member and the at least one coil surrounding the second core member, using the determined spacing, so that a magnetic field of predetermined intensity will be applied to a central portion of the treatment object;
applying the amount of current to the at least one coil surrounding the first core member and the at least one coil surrounding the second core member while the treatment object is positioned between the end face of the first core member and the end face of the second core member to energize the at least one coil surrounding the first core member and the at least one coil surrounding the second core member and produce the magnetic field of predetermined intensity which is applied to the central portion of the treatment object.

19. The method according to claim 18, further comprising reducing the spacing between the end face of the first core member and the end face of the second core member until the end face of the first core member and the end face of the second core member both contact the treatment object, the reducing of the spacing occurring before the amount of current is applied to the at least one coil surrounding the first core member and the at least one coil surrounding the second core member.

20. The method according to claim 18, wherein an intensity of the magnetic field at a middle portion between the end face of the first core member and the end face of the second core member is in the range of 30 to 1000 mT.

21. The method according to claim 18, further comprising adjusting the spacing between the end face of the first core member and the end face of the second core member method so the spacing is within a range of 20 to 200 mm.

22. The method according to claim 18, further comprising holding the first and second core members with a force greater than a magnetic force acting between the end face of the first core member and the end face of the second core member.
